SEQUENCE LISTING (5 sequences)

SEQ ID NO: 1 - DNA, 1146 bp, Homo Sapiens
ctggtcctgtcctgttctccagcatggtgtgtctgaagctccctggaggctcctgcatggcagctctgacagtgacactgatggtgctgagctccccactggctttggctggggacacccaaccacgtttcctgtggcagggtaagtataagtgtcatttcttcaacgggacggagcgggtgcagttcctggaaagactcttctataaccaggaggagttcgtgcgcttcgacagcgacgtgggggagtaccgggcggtgacggagctagggcggcctgtcgccgagtcctggaacagccagaaggacatcctggaggacaggcggggccaggtggacaccgtgtgcagacacaactacggggttggtgagagcttcacagtgcagcggcgagtccatcctgaggtgactgtgtatcctgccaagactcagcccctgcagcaccacaacctcctggtctgctctgtgagtggtttctatccaggcagcattgaagtcaggtggttccggaacggccaggaagagaaggctggggtggtgtccacaggcctgatccagaatggagactggaccttccagaccctggtgatgctggaaacagttcctcggagtggagaagtttacacctgccaagtggagcacccaagtgtgatgagccctctcacagtggaatggagagcacggtctgaatctgcacagagcaagatgctgagtggtgtcgggggctttgtgctgggcctgctcttccttggggccgggttgttcatctacttcaggaatcagaaaggacactctggacttcagccaacaggattcctgagctgaagtgaagatgaccacattcaaggaaaaaccttctgccccagctttgcaggatgaaacacttcccgcttggctctcattcttccacaagagagacctttctccggacctggttgctactggttcagcaactgcagaaaatgtcctcccctgtggctgcctcagctcatgcctttggcctgaagtcccagcattgatggcagcccctcatcttccaagttttgtgctcccctttacctaacgcttcctgcctcccatgcatctgtactcctcctgtgccacaaacacattacattattaaatgtttctcaaacatggagttaaa

SEQ ID NO: 2 - DNA, 2326 bp, Homo Sapiens
agccagtcctgagaggaaaggaagtataatcaatttgttattaactgatgaaagaattaagtgaaagataaaccttaggaagcagagggaagttaatctatgactaagaaagttaagtactctgataactcattcattccttcttctgttcatttacattatttaatcacaagtccatgatgtgccaggcactcaggaaatagtgaaaatcggacacgcgatattctgcccttgtgtagcacacactgtagtgggaaagaaagtgcacttttaactggacaactatcaacacgaagaggggaggaagcaggggctggaaatgtccacagactttgccaaagacaaagcccataatatttgaaagtcagtttcttccatcattttgtgtattaaggttttttattctcctgttctctgccttcctgcttgtcatcttcactcatcagctgaccacgttgcctcttacggtgtaaacttgtaccagtcttacggtccctctggccagttcacccatgaatttgatggagacgaggagttctatgtggacctggagaggaaggagactgtctggaagttgcctctgttccacagacttagatttgacccgcaatttgcactgacaaacatcgctgtgctaaaacataacttgaacatcctgattaaacgctccaactctaccgctgctaccaatggtatgtgtccaccattctgcctttctttactgatctatccctttataccaagtttcattattttctttccaagaggtccccagatcttctcatggcaattgctgaaattttatcatttctcatctctaaaatcacatatccccatgtaatacaagggtctttccattatgcattcattaaatcattctaggagaggtctcatcaacctcctactttattaaacatgcccacagagagaagggcacaggagtaaagcagaggcaatgtgtcattgctcccaagtagaaggtaaataaggcctctttgaccagcaggagaggaaatgctggtaggaagactcttccaggatgtaatgcagaagctcagggcagagctattcacacttcacaccagtgctgtttcctcaccatagaggttcctgaggtcacagtgttttccaagtctcccgtgacactgggtcagcccaacaccctcatctgtcttgtggacaacatctttcctcctgtggtcaacatcacctggctgagcaatgggcactcagtcacagaaggtgtttctgagaccagcttcctctccaagagtgatcattccttcttcaagatcagttacctcaccttcctcccttctgctgatgagatttatgactgcaaggtggagcactggggcctggatgagcctcttctgaaacactggggtaaggatgagtttcaccattttttgatgctttcttgtctgtcaagttcagaacttcctgccttttactctatgtcccaaaacttgttttccacacttcatgagtttcttttatcttttttttttttgaaagaattaagcaacaaaagcacagatttattaaaaaagaaagtacactccacagggtgggagcaggcctgccacttcatgggtttctaataacagacttcactctcctccctgagccaggggccttgagtctttgcagagccaaccctccaccccatcccatccctcacacatgcacatgagcacactctgcattctgacctcaacaacttcacttccacagagcctgagattccagcacctatgtcagagctcacagagactgtggtctgtgccctggggttgtctgtgggcctcgtgggcattgtggtggggaccgtcttgatcatccgaggcctgcgttcagttggtgcttccagacaccaagggcccttgtgaatcccatcctgaaaaagaaggtaagtttgagatttgttagagctgaagctgcaggaaggaaagtgggaggaggctgtggacatgaatgtggttgaaagttgtaggggaattgggaagtggcatgatgatgacacaggagcccccttggacccatcgatctcatgtctgtcctgttgcaggtgcatcaccatctacagcagcggaagagtggacttgctacatgacctagcactattctctggcccgatttatcataccctttttctcctgcaaatgtttctcctcttaccttttctctgcttttttttccttaagcttctgtatcccctcagagctcacaaatgcctttgaattctttccctgacctcctgatttttttttcttttctcaggtgttacctactaagagatgcctggggtaagccgcccagctac

SEQ ID NO: 3 - DNA, 240 bp, Homo Sapiens
aattaggacatgacataggtggttagcaaataaagaatatttgcagagtaaagcaaaatgtgcagtgaggattggaggagctgggcatttagactgatagagacataatgcctggttttccattcctgtgcctggatagtattaaaagcacaaataaaaagctatcctgctgctatctgcctgctattcatattatagaagagatggagactgtcagagagatctattatgccaactctg

SEQ ID NO: 4 - DNA, 201 bp, Homo Sapiens
attaatttatgaagagaaccttttctctttctactagacaaaggtaacaaattagcttttgttaaaatggcatactgttcttctgagtttcatttacttactactttaccttacattaaaattatgacctgaagacagaaggaactggaacaatgttcactgtgttatggccggatgaagcaggaagaggagaagagacaa

SEQ ID NO: 5 - DNA, 801 bp, Homo Sapiens
cccctaaagctcaagaaagttgtctgggatatacaaagtactgactacaccaattattatatcatgatctaaagtgtaagtcacaagtcaaactcttgatcacttcattcatattacatgttccagattataaccactggaaaacttctctaacagtaattttgcatgaactcatgtgttctaaaactacaaccaagtgtgcatttcttcctgataggcatttaacacactaagtgaccatatgacttaaattgtccatatgatgctgagagttataagattcatagactcttaagtttgggcataccaaccacaatccatcatgcaattaaagcagtacatttgaaaccagacttcagcaggtttaaaaactaggtggttgttctggtttggtgatagagactacaatcccagaagggtttatgctcttgtttatcttctccttgatggagtataattgttttagtgtctactgaattttcatctaatttggtcttgaggtctctctcatgagaacagctataaactagttttgctctgctggagctttagggaatttgggtgtggagtttacaatgtatcttcatgggatgcttctttattctggaagatggtctaatgcctaagtgtctgacctgtaaccaagcgcccctctcacaggaaacttgtttatactggaagacacttttgtggctcttgtctgacctgtgtccagtttattcctgccaaaatttcactctctgggacagctctgtctgggaaagaagttaaatttgaaggtgtcaggtaagatagaaagaa

(19) United States
(12) Patent Application Publication
(10) Pub. No.: US 2009/0036424 A1
(43) Pub. Date: Feb. 5, 2009

(54) ASSOCIATION BETWEEN HLA-DRBI*07 ALLELE AND SUSCEPTIBILY TO INCREASED LEVELS OF ALAT FOLLOWING XIMELAGATRAN ADMINISTRATION

(75) Inventors: Olof Bengtsson, Mölndal (SE); Ellen Brown, Cheshire (GB); Stefan Carlsson, Molndal (SE); Neil James Gibson, Cheshire (GB); Ansar Jawaid, Cheshire (GB); Andreas Kindmark, Molndal (SE); Ruth Eleanor March, Cheshire (GB)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/067,867

(22) PCT Filed: Oct. 3, 2006
(86) PCT No.: PCT/GB2006/003661

§ 371 Date: Mar. 24, 2008

(30) Foreign Application Priority Data
Oct. 5, 2005 (GB) 0520235.3

(51) Int. Cl. A61K 31/397; C12Q 1/68

(52) U.S. Cl. 514/210.17; 435/6

(57) ABSTRACT

This invention relates to a method for administering a pharmaceutically useful anticoagulant drug to certain suitable patients and a method for identifying those patients suitable for receiving the drug. In particular, the invention surrounds the identification of an association between HLA-DRB1*07 allele and susceptibility to increased levels of alanine aminotransferase (ALAT) following ximelagatran administration. Thus, this invention relates to methods for predicting susceptibility to elevated ALAT following ximelagatran administration and to methods for administering a pharmaceutically useful anticoagulant drug to certain suitable patients.

### FIELD OF THE INVENTION

This invention relates to a method for administering a pharmaceutically useful anticoagulant drug to certain suitable patients and a method for identifying those patients suitable for receiving the drug. The inventors have found an association between the existence of the HLA-DRB1*07 allele and elevated ALAT following ximelagatran administration. This association also exists with genetic markers that predict the presence of the HLA-DRB1*07 allele, such as the HLA-DQA1*02 allele and three specific single nucleotide polymorphisms close to the human DRB-1 gene. Thus, in particular, this invention relates to a method for administering a pharmaceutically useful anticoagulant drug to certain suitable patients and a method for identifying those patients suitable for receiving the drug.

### BACKGROUND

Blood coagulation is the key process involved in both haemostasis (i.e. the prevention of blood loss from a damaged vessel) and thrombosis (i.e. the formation of a blood clot in a blood vessel, sometimes leading to vessel obstruction).

Coagulation is the result of a complex series of enzymatic reactions. One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V and factor VIII leading to a “positive feedback” generation of thrombin from prothrombin.

By inhibiting the aggregation of platelets and the formation and crosslinking of fibrin, effective inhibitors of thrombin would therefore be expected to exhibit antithrombotic activity. In addition, antithrombotic activity would be expected to be enhanced by effective inhibition of the positive feedback mechanism.

The development of low molecular weight inhibitors of thrombin has been described by Claesson (Blood Coagul. Fibrin. 5:411, 1994), and certain thrombin inhibitors based on peptide derivatives have been disclosed, for example, in European Patent Application 0 669 317 and International Patent Applications WO 95/23609, WO 95/35309, WO 96/25426 and WO 94/29336.

The latter application discloses the peptide derivatives R^{a}OOC—CH₂—(R)Cgl-Aze-Pab-H, wherein R^{a} represents H, benzyl or C₁₋₆ alkyl. When R^{a} represents H the compound is known as melagatran.

The compound known as ximelagatran (EtOOC—CH₂—(R)Cgl-Aze-Pab-OH) has been developed for use, for example, in orthopaedic surgery and in atrial fibrillation. Upon oral administration ximelagatran is metabolised to the active thrombin inhibitor melagatran. Further details on ximelagatran and its preparation are contained in, for example, WO 97/23499.

For reference, Aze=S-Azetidine-2-carboxylic acid; Cgl=cyclohexylglycine;
H-Pab-H=1-amidino-4-aminomethyl benzene; Pab-OH=4-aminomethyl-benzamidoxime (4-aminomethyl-1-(amino-hydroxyiminomethyl)benzene).

Phase III clinical trials have been performed using fixed doses of melagatran and ximelagatran for the prevention of VTE in hip or knee replacement surgery. In addition, clinical trials have been performed using ximelagatran for the treatment and long-term secondary prevention of VTE, and for the prevention of stroke in patients with non-valvular atrial fibrillation. Ximelagatran has also been tested for secondary thrombosis prophylaxis post-myocardial infarction/acute coronary syndrome (ACS).

Alanine aminotransferase (ALAT) is an enzyme mostly expressed in the liver (EC 2.6.1.2). It is also called serum glutamate pyruvate transaminase (SGPT) or alanine transaminase (ALT). This enzyme is released into the plasma by liver cell death, which is a normal event. However, when liver cell death increases, ALAT levels rise above the normal range. The spill-over of this enzyme into blood is routinely measured as a marker of abnormal liver-cell damage. For example, alcoholic or viral hepatitis will increase ALAT levels, as will severe congestive heart failure. An elevated ALAT in the presence of normal levels of plasma alkaline phosphatase helps distinguish liver disease caused by liver-cell damage from diseases caused by problems in biliary ducts.

Elevations of ALAT are normally measured in multiples of the upper limit of normal (ULN), with a reference range of 15-45 U/L in most laboratories. In 1987, in a study of 19,877 healthy Air Force recruits, only 99 (0.5%) had confirmed ALAT elevations (as reviewed in Green & Flamm (2002) Gastroenterology 123:1367-1384).

ALAT is markedly raised in hepatitis and other acute liver damage. The enzyme aspartate transaminase (AST) has a similar role, but this enzyme tends to be found in other tissues, such as the heart, so is not as specific to the liver.

During longer-term treatment with ximelagatran (>35 days) 7.9% of patients exhibited levels of alanine aminotransferase (ALAT) 3-fold or more above the upper limit of normal (≧3×ULN), compared with 1.2% in the comparator groups. The increase in ALAT values with ximelagatran usually occurred within the first 6 months of treatment and were mainly asymptomatic. Furthermore, these increases in ALAT were reversible in most patients regardless of whether treatment was continued or discontinued. Subject to the future regulatory approval of ximelagatran, regular liver function testing (LFT) using an appropriate algorithm may be required if ximelagatran is used for treatment periods exceeding a month. Studies are currently ongoing to try and establish the mechanism of the ALAT elevations, and their hepatic and overall clinical significance.

Accordingly, it is desirable to identify which patients are likely to experience raised ALAT levels when receiving ximelagatran. The identification of biomarkers of drug efficacy or safety, can be used to design diagnostic or prognostic tests.

The invention also relates to methods and materials for stratifying patients to be treated with ximelagatran into those that are at high or low likelihood of experiencing elevated ALAT levels following ximelagatran treatment, thus offering the ability to make informed decisions about whether or not a particular patient or sub-patient population should be treated with the drug.

The sub-groups of individuals identified as having increased or decreased likelihood of experiencing elevated ALAT following ximelagatran administration, can be used, inter alia, for targeted clinical trial programs and possibly also pharmacogenetic therapies.

By elevated ALAT we mean, for example ≧3-fold upper limit of normal (as reviewed in Green & Flamm, ibid).

The present invention is based on the finding of an association between HLA DRB1*07 alleles and raised (≧3×ULN) ALAT levels following ximelagatran treatment.

The HLA-DRB1 gene is part of the major histocompatibility complex (MHC), which was first identified in mice by Peter Gorer in 1936. In the 1950s and 1960s, studies of human leukocyte antigens, particularly by Dausset, Payne and van Rood, first identified the human homologue of the mouse MHC, now known as the human leucocyte antigen (HLA) complex and demonstrated its major role in transplant survival (reviewed by Sharz & Shreffler, in Clinical Immunology, ed. Parker, W. B. Saunders Company (1980)).

HLA-DRB1 belongs to the HLA class II beta chain paralogues located on human chromosome 6. The class II molecule is a heterodimer consisting of an alpha (DRA) and a beta chain (DRB), both anchored in the membrane. It plays a central role in the immune system by presenting peptides derived from extracellular proteins. Class II molecules are expressed in antigen presenting cells (APC: B lymphocytes, dendritic cells, macrophages). The beta chain is approximately 26-28 kDa. It is encoded by 6 exons, exon one encodes the leader peptide, exons 2 and 3 encode the two extracellular domains, exon 4 encodes the transmembrane domain and exon 5 encodes the cytoplasmic tail. Within the DR molecule the beta chain contains all the polymorphisms specifying the peptide binding specificities. Hundreds of DRB1 alleles have been described and typing for these polymorphisms is routinely done for bone marrow and kidney transplantation. DRB1 is present in all individuals.

The HLA antigens were first detected using microcytoxicity assays in which sera could be tested against lymphocytes. Originally each laboratory working in the HLA system used its own nomenclature, and it was not until a World Health Organization (WHO) nomenclature committee was convened (1967) that common terms were agreed. At this time antigens in the HLA-D or DR locus were detected by cellular and serological methods. Examples of alternative names for the antigen DR7 were DRw7, Dw7, UK7, FT1, etc. (Bodmer, British Medical Bulletin 34, 233-240 (1978)).

HLA nomenclature was further standardised in 2002 (Marsh et al, Human Immunology, 63, 1213-1268 (2002)). According to this nomenclature, the so-called low-resolution type or two-number code (in this case HLA-DRB1*07) corresponds to all the alleles that encode the DR7 antigen. The four-number codes (HLA-DRB1*0701, 0703, 0704 etc.) refer to specific DRB1*07 alleles.

The first sequence of DRB1*07 was published in 1986 (Gergersen et al. Proc. Natl. Acad. Sci. U.S.A. 83:9149-9153, 1986) and referred to as DR7. Gregersen et al noted that nucleotide sequence in the first domain of the HLA-DR beta chains diverged up to 11% between DR4, DR7 and DR9 haplotypes and that most of this variation resulted in productive amino acid changes (Gregersen et al, ibid). Young et al., sequenced DRB1*0701 from the cell line MANN, homozygous for HLA region genes and serologically identified as DR7, DRw53, DQw2, DP1 (Young et al. Proc. Natl. Acad. Sci. U.S.A. 84:4929-4933, 1987). (EMBL Accession number: HS09201; SEQ ID NO: 1, herein). A number of other DRB1*07 alleles (also known as subtypes) are now known (reviewed in Schreuder et al, Human Immunology 66:170-210, 2005).

The reported frequency of DRB1*07 ranges from 27% in the Basque population (Comas, D., et al. Annals of Human Genetics, 62, 123, 1998) to zero in the Nuba population of Sudan and the Hoton population in Mongolia (Clayton et al. In: Genetic Diversity of HLA: Functional and Medical Implications (ed. by D. Charron), Vol. 1, p. 665. EDK, Paris, 1997). The frequency in two Japanese populations was reported as 0.2% and 0.4% (Lin et al., Tissue Antigens, 50:507, 1997; and Wang et al., Tissue Antigens, 41:223. 1993). The average frequency in North European populations (Norwegian, German, and Polish) was reported as 12% (reviewed in Gilbert and Sanchez-Masaz, European Journal of Immunogenetics 30, 361-374, 2003). Interestingly, this distribution mirrors the frequency of cases of ximelagatran induced raised ALAT in North Europeans as opposed to Asians observed by the inventors.

The use of knowledge of polymorphisms to help identify patients most suited to therapy with particular pharmaceutical agents is often termed “pharmacogenetics”. Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43:254; Marshall (1997), Nature Biotechnology. 15:1249; International Patent Application WO 97/40462, Spectra Biomedical; and Schafer et al, (1998), Nature Biotechnology. 16:33.

Clinical trials have shown that patient response to treatment with pharmaceuticals is often heterogeneous. Thus, there is a need for improved approaches to pharmaceutical agent design and therapy. Furthermore, there may be opportunities for clinical trial patient selection.

### DISCLOSURE OF THE INVENTION

According to a first aspect of the invention there is provided a method of determining an individual's likelihood of experiencing elevated ALAT levels following ximelagatran administration, comprising determining the HLA-DRB1*07 status of the individual, and assessing the likelihood according to whether or not the individual has an HLA-DRB1*07 allele.

The HLA-DRB1*07 status simply means determining whether the individual has none, one or two copies of the HLA-DRB1*07 allele. This can be determined by various methodologies known to the person skilled in the art. These include, for example, serological or immunological methods to detect the presence of the DR7 antigen or molecular biological techniques to determine the nucleic acid sequence corresponding to the HLA-DRB1*07 alleles (e.g. SEQ ID NO: 1, or a subtype sequence thereof).

In one embodiment, the HLA-DRB1*07 status is determined by detection of the HLA-DRB1*07 allele on one or both copies of the chromosome 6, via nucleic acid determination, in a sample that has previously been removed from the individual.

According to another aspect of the invention there is provided a method for screening an individual for a genetic predisposition to release elevated ALAT from the liver following ximelagatran administration, comprising analysing the individual's nucleic acid in a sample removed from the individual for the presence or absence of an HLA-DRB1*07 allele, and determining the status of the human by reference to the presence or absence or one of both possible copies of an HLA-DRB1*07 allele.

According to another aspect of the invention there is provided a method of determining whether or not a patient in need of treatment with ximelagatran has a genetic makeup indicative of subjects likely to exhibit elevated ALAT following ximelagatran treatment, the method comprising:
(a) determining whether or not the patient's genome has an HLA-DRB1*07 allele; and,
(b) if the patient's genome has one or both copies of an HLA-DRB1*07 allele identifying that patient as one at increased likelihood of exhibiting elevated ALAT following ximelagatran treatment.

In one embodiment of the invention the HLA-DRB1*07 allele determination is based on detection of the presence of the sequence disclosed in SEQ ID NO: 1, or a sequence that encodes a polypeptide with up to 4 amino acid differences provided the encoded allele retains the same antigenic or binding specificity or function as that encoded by SEQ ID NO: 1.

In alternate embodiments of this aspect the HLA-DRB1*07 allele determination is based on detection of the presence of a sequence with up to 3 or up to 2, or up to 1 encoded amino acid differences provided the encoded allele retains the same antigenic or binding specificity or function as that encoded by SEQ ID NO: 1.

According to another aspect of the invention there is provided a method for screening an individual for a genetic predisposition to release elevated ALAT from the liver following ximelagatran administration, comprising detecting the presence or absence of the DR7 antigen in a sample removed from the individual, and determining the status of the human by reference to the presence or absence of the DR7 antigen. In certain embodiments, detection of the DR7 antigen is carried out using cellular, serological or immunological techniques.

The HLA-DQA1 gene is also part of the major histocompatibility complex (MHC), and lies close (47 kb) to HLA-DRB1 on human chromosome 6. It encodes the alpha chain of the HLA-DQ heterodimer and plays a similar role to HLA-DR in the immune system by presenting peptides derived from extracellular proteins. Unlike the DR molecule, both the alpha chain and the beta chain of the DQ heterodimer are polymorphic and contain alleles that specify the peptide binding specificities of the molecule. Six common DQA1 alleles have been described. DQA1 is present in all individuals.

The inventors have also determined a statistically significant association between the DQA1*02 allele (SEQ ID NO: 2; EMBL Accession number AY375912) and cases (patients with elevated ALAT levels), and that this allele predicts the presence of the DRB1*07 allele by virtue of its tight linkage disequilibrium with DRB1*07 (D′=0.98). Thus, alleles of the DQ molecule that are in linkage disequilibrium with DRB1*07 alleles with D′>0.9 also form part of this invention.

Single nucleotide polymorphisms (SNPs) represent one of the most common forms of genetic variation. These polymorphisms appear when a single nucleotide in the genome is altered (such as via substitution, addition or deletion). For example, if at a particular chromosomal location one member of a population has an adenine and another member has a thymine at the same position, then this position is a single nucleotide polymorphic site. Each version of the sequence with respect to the polymorphic site is referred to as an “allele” of the polymorphic site. SNPs tend to be evolutionarily stable from generation to generation and, as such, can be used to study specific genetic abnormalities throughout a population. If SNPs occur in the protein coding region it can lead to the expression of a variant, sometimes defective, form of the protein that may lead to development of a genetic disease. Such SNPs can therefore serve as effective indicators of the genetic disease. Some SNPs may occur in non-coding regions, but nevertheless, may result in differential or defective splicing, or altered protein expression levels. SNPs can therefore be used as diagnostic tools for identifying individuals with a predisposition for certain diseases, genotyping the individual suffering from the disease in terms of the genetic causes underlying the condition, and facilitating drug development based on the insight revealed regarding the role of target proteins in the pathogenesis process.

The inventors have also determined a statistically significant association between SNPs close to the DRB1 gene (SNPs rs2858869, rs17426385 and rs9275141) and cases (patients with elevated ALAT levels), and that these polymorphisms are in linkage disequilibrium with DRB1*07 (D′=0.85, 1.00 and 0.69 respectively). Thus, polymorphisms that are in linkage disequilibrium with DRB1*07 alleles with D′>0.68 also form part of this invention.

The presence of a G allele at position 101 of SEQ ID NO: 3 could also be used to optimise the benefit: risk ratio of an individual for ximelagatran treatment. Thus, the methods and kits of the invention can also be adapted or used for detecting the presence of rs2858869.

The presence of a C allele at position 101 of SEQ ID NO: 4 could also be used to optimise the benefit: risk ratio of an individual for ximelagatran treatment. Thus, the methods and kits of the invention can also be adapted or used for detecting the presence of rs17426385.

The presence of a G allele at position 401 of SEQ ID NO: 5 could also be used to optimise the benefit: risk ratio of an individual for ximelagatran treatment. Thus, the methods and kits of the invention can also be adapted or used for detecting the presence of rs9275141.

The location of a polymorphisms can be precisely mapped by reference to published EMBL (or other sequence database) sequence accession numbers (i.e. see above), alternatively, the person skilled in the art can precisely identify the location of the rs2858869, rs9275141 and rs9275141 polymorphisms close to the DRB1 gene simply by provision of flanking sequence adjacent the polymorphism sufficient to unambiguously locate the polymorphism (e.g. position 101 of SEQ ID NO: 3, position 101 of SEQ ID NO: 4 or position 401 of SEQ ID NO: 5, herein). Provision of 10 or more nucleotides each side of the polymorphism should be sufficient to achieve precise location mapping of the particular polymorphism.

SEQ ID No: 3, derives from EMBL accession number AL662789. The SNP at position 101 being G is in reverse orientation.
SEQ ID No: 4, derives from EMBL accession number AY663414. The SNP at position 101 being C is in forward orientation.
SEQ ID NO: 5, derives from EMBL accession number AY663413, The SNP at position 401 being G is in forward orientation.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas. The presence of a particular base at a polymorphism position will be represented by the base following the polymorphism position. For (a hypothetical) example, the presence of adenine at position 300 will be represented as: 300A.

According to another aspect of the invention there is provided a method for genotyping the DRB1 gene in a human individual comprising the steps of:
a) treating nucleic acid from a sample that has been removed from the individual so as to identify the nucleotides present corresponding to the *07 allelic sequences of the DRB1 gene, and
b) assigning the individual a particular genotype according to the nucleotides detected in step a).

According to another aspect of the invention there is provided a method of identifying a subtype of patients likely to experience elevated ALAT when treated with ximelagatran, comprising determining the HLA DRB-1*07 allele status of each patient, and identifying patients that possess an DRB-1*07 allele as those that are likely to experience elevated ALAT when treated with ximelagatran.

The test sample (the nucleic acid containing sample) is conveniently a sample of blood, plasma, bronchoalveolar lavage fluid, saliva, sputum, cheek-swab or other body fluid or tissue (such as a biopsy sample) obtained from an individual that contain nucleic acid molecules. The nucleic acid containing sample that is to be analysed can either be a treated or untreated biological sample isolated from the individual. A treated sample, may be for example, one in which the nucleic acid contained in the original biological sample has been isolated or purified from other components in the sample (tissues, cells, proteins etc), or one where the nucleic acid in the original sample has first been amplified, for example by polymerase chain reaction. Thus, it will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation.

For the avoidance of doubt, the methods of the invention do not involve diagnosis practised on the human body. The methods of the invention are preferably conducted on a sample that has previously been removed from the individual. The kits of the invention, however, may include means for extracting the sample from the individual.

When specifying a particular nucleotide at an allele position it is important to appreciate which of the two complementary strands of nucleic acid the nucleotide resides on. For example, a G on the positive strand will correspond to a C on the negative (reverse) strand. The SNP alleles referred to herein, including the claims, are defined by the assays described in Table 1. For example, rs2858869 is on the reverse strand. The correct strand may also be deduced by the nucleotide sequence adjacent the allele, by reference to the sequence listings provided herein.

The ability to identify patients that have increased likelihood of experiencing elevated ALAT following ximelagatran treatment allows the patient or their physician to assess their suitability for treatment with ximelagatran. It also allows, for example, the option to include or exclude such individuals in clinical studies.

According to another aspect of the invention there is provided a method for selecting subjects for inclusion in a clinical study of a therapeutic agent comprising:
(a) determining the HLA-DRB1*07 status of each subject;
(b) grouping the individuals according to the heterozygous, homozygous positive or homozygous negative HLA-DRB1*07 status that each individual has; and,
(c) selecting subjects in one or more groups for inclusion in a clinical study of the therapeutic agent.

In a particular embodiment, the therapeutic agent is ximelagatran. In another embodiment, the homozygous negative subjects are included in a clinical study of ximelagatran.

The presence of one or two copies of HLA-DRB1*07, however, does not mean that the individual will experience elevated ALAT following ximelagatran treatment. It merely suggests that the individual compared to the population as a whole has a higher likelihood of experiencing elevated ALAT.

According to a further aspect of the invention there is provided a diagnostic or prognostic method of predicting susceptibility to elevated ALAT following ximelagatran administration, based on the detection of the presence or absence of HLA-DRB1*07, or a polymorphism in linkage disequilibrium with a D′>0.8 therewith, in an individual.

According to a further aspect of the invention there is provided a diagnostic or prognostic method of predicting susceptibility to elevated ALAT following ximelagatran administration, based on the detection of the presence or absence of HLA-DRB1*07, or a polymorphism in linkage disequilibrium with a D′>0.68 therewith, in an individual.

In a particular embodiment the polymorphism is in linkage disequilibrium with a D′>0.8. In a further particular embodiment the polymorphism is in linkage disequilibrium with a D′>0.9.

According to a further aspect of the invention there is provided a method of predicting susceptibility to elevated ALAT following ximelagatran administration, in an individual, comprising determining the presence or absence in a sample from said individual of HLA-DRB1*07, or a polymorphism in linkage disequilibrium with a D′>0.68 therewith, wherein the presence of said allele or polymorphism is predictive of elevated ALAT following ximelagatran administration.

In a particular embodiment the polymorphism is in linkage disequilibrium with a D′>0.8. In a further particular embodiment the polymorphism is in linkage disequilibrium with a D′>0.9.

In one embodiment, determination of the presence of HLA-DRB1*07 is based on determination whether or not the individual has a sequence according to SEQ ID NO: 1. In another embodiment of the invention, determination of the presence of HLA-DRB1*07 is based on determination whether or not the individual has a sequence according to SEQ ID NO: 2 (DQA1*02 allele).

In view of the statistically significant association between the G nucleotide at rs2858869, the C nucleotide at rs17426385 or the G nucleotide at rs9275141 (positions 101, 101 or 401 of SEQ ID NO: 3, SEQ ID NO: 4 or SEQ ID NO: 5, respectively), and elevated ALAT in patients on ximelagatran treatment, the methods, uses, kits and nucleic acids of the invention are equally applicable to determination of these SNPs on their own, or in conjunction with determination of HLA-DRB1*07 status.

Thus, according to another aspect of the invention there is provided a method of diagnosing or predicting susceptibility to elevated ALAT following ximelagatran administration in an individual, comprising determining the presence or absence in a sample removed from said individual of a guanine (G) nucleotide at rs2858869, a C nucleotide at rs17426385 or a G nucleotide at rs9275141 (positions 101, 101 or 401 of SEQ ID NO: 3, SEQ ID NO: 4 or SEQ ID NO: 5, respectively), wherein the presence of said nucleotide is diagnostic or predictive of susceptibility to elevated ALAT following ximelagatran administration. Clearly, as there are two complementary strands of nucleic acid, determination of G at position 101 of SEQ ID NO: 3, for example, could be carried out indirectly by determination of the presence of C on the complementary strand.

According to another aspect of the invention there is a provided a method for determining whether or not a patient to be treated with ximelagatran is likely to experience elevated ALAT, comprising determining the identity of nucleotides at rs2858869, rs17426385 or rs9275141 (positions 101, 101 or 401 SEQ ID NO: 3, SEQ ID NO: 4 or SEQ ID NO: 5, respectively), wherein if the patient's genome comprises a G, C or G. respectively at said position they are likely to experience elevated ALAT.

In another aspect the diagnostic or prognostic kits and methods of the invention involve detection of these SNPs as well as the presence of DQA1*02 and DRB1*07 (directly or indirectly).

The SNPs, DQA1*02 and DRB1*07 alleles of the invention demonstrate significant association to experiencing elevated ALAT following ximelagatran administration. However, the person skilled in the art will appreciate that a diagnostic test consisting solely of the SNPs or allelic detection of the invention may not be diagnostic of the occurrence of elevated ALAT following ximelagatran administration for any particular individual. Nevertheless, in line with future developments we envisage that the methods and kits of the present invention could form part of a panel of markers that in combination will be predictive of elevated ALAT following ximelagatran administration for an individual, within normal clinical standards sufficient to influence clinical practice.

Because there are two copies of each chromosome (a maternal and paternal copy), at each chromosomal location the human may be homozygous for an allele or the human may be a heterozygote. If the individual is heterozygous the presence of both alternate polymorphisms will be present. With regard to HLA DRB1*07 status, persons with two copies of this allele are referred to as homozygous positives, those with none are homozygous negatives and those with one copy are heterozygotes.

A wide variety of methods are available for determining the DRB1*07/DR7 or DQA1*02 carrier status of an individual. The person skilled in the art is at liberty to choose any appropriate methodology. Representative examples of available methods include:

1. The AlleleSEQR DRB1 Typing kit (Atria Genetics, U.S.A.), distributed by Abbott Diagnostics. This method uses the Connexia Genomics Assign-SBT™ 3.2.7 software to call alleles. (Sayer D C et al. Tissue Antigens 63:412-23, 2004).
2. Serological and molecular typing kits from Dynal Biotech, part of Invitrogen Corporation, Carlsbad, Calif. 92008, USA. Dynal kits that utilise SSP typing are available for both DR and DQ low resolution typing (Dynal AllSet+™ SSP kit). This technique uses Sequence Specific Primers for DNA based Tissue Typing. Each SSP kit consists of a panel of primer mixes that each contain a specific primer pair (i.e., the allele- and the group-specific primers) as well as a control primer pair that matches non-allelic sequences present in all samples. This control acts as an internal PCR control to verify the efficiency of the PCR amplification.
3. Pyrosequencing (Entz P et al. Tissue Antigens 65:67-80 (2005), Casamitjana N et al. Hum Immunol. 66:85-91, 2005).
4. Sequence-specific primer (SSP)—PCR: Olerup O and Zetterquist H. (Tissue Antigens 39: 225-235, 1992), Bunce M et al. Tissue Antigens 46: 355-367, 1995)
5. Sequence specific oligonucleotide probe (SSOP) assay: (Fischer et al, Tissue Antigens 55:166-70, 2000).

### 6. Serology (Dynal Biotech, Invitrogen Corporation, Carlsbad, Calif. 92008, USA, Biologische Analysensystem GmbH, D-35423 Lich, Germany, World Marrow Donor Association Quality Assurance and IT Working Groups (Hum Immunol. 66:170-210, 2005).

7. Direct sequence analysis (e.g. dideoxy sequencing) for the sequence depicted in SEQ ID NO: 1 for DRB1*07 detection, or SEQ ID NO: 2 for DQA1*02 detection, or a sub-type variant sequence thereof that encodes a protein with the same specificity or function thereof.

It will be apparent to the person skilled in the art that the sequence conferring the specificity of HLA-DRB1*07 depends on the nucleotides of the hypervariable region in exon 2 of the DRB1 gene (more than 95 nucleotides). Because of the hundreds of possible alleles present in an individual in this region, sequencing or genotyping of all the polymorphic residues within this exon may be necessary in order to assign the allelic status of the individual. Analytical procedures that detect the high resolution or DRB1*07 alleles of the individual (for example, HLA-DRB1*0701, 0703, 0704 etc) can also be used to assign DRB1*07 status, since DRB1*07 status encompasses all these alleles. In addition, analytical procedures that detect the presence of the DR7 antigen may also be used to assign HLA-DRB1*07 status.

It will be apparent to the person skilled in the art that there are a large number of analytical procedures, which may be used to detect the presence or absence of variant nucleotides at one or more polymorphic positions of the invention. In general, the detection of allelic variation requires a mutation discrimination technique, optionally an amplification reaction and optionally a signal generation system. List 1 lists a number of mutation detection techniques, some based on the PCR. These may be used in combination with a number of signal generation systems, a selection of which are listed in List 2. Further amplification techniques are listed in List 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| Bp | base pair |
| CMC | Chemical mismatch cleavage |
| COPS | Competitive oligonucleotide priming system |
| DGGE | Denaturing gradient gel electrophoresis |
| ELISA | Enzyme Linked ImmunoSorbent Assay |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

### List 1—Mutation Detection Techniques

General: DNA sequencing, Sequencing by hybridisation
Scanning: PTT, SSCP, DGGE, TGGE, Cleavase, Heteroduplex analysis, CMC, Enzymatic mismatch cleavage

### Hybridisation Based

Solid phase hybridisation: Dot blots, MASDA, Reverse dot blots, Oligonucleotide arrays (DNA Chips).

Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)

Extension Based: ARMS™, ALEX™—European Patent No. EP 332435 B1 (Zeneca Limited), COPS—Gibbs et al (1989), Nucleic Acids Research, 17, 2347.

### Incorporation Based Mini-sequencing, APEX

Restriction Enzyme Based: RFLP, Restriction site generating PCR

### Ligation Based: OLA

Other: Invader assay

### List 2—Signal Generation or Detection Systems

Fluorescence: FRET, Fluorescence quenching, Fluorescence polarisation—United

### Kingdom Patent No. 2228998 (Zeneca Limited)

Other: Chemiluminescence, Electrochemiluminescence, Raman, Radioactivity, Colorimetric, Hybridisation protection assay, Mass spectrometry

### List 3—Further Amplification Methods

SSR, NASBA, LCR, SDA, b-DNA
List 4—Protein variation detection methods

### Immunoassay

### Immunohistology

Peptide sequencing

In one embodiment, the presence or absence of a particular SNP or allele is determined using polymerase chain reaction (PCR). In a particular SNP discriminating embodiment the PCR is performed with allele-specific oligonucleotide primers capable of discriminating between the different bases at a particular allele. Such as using amplification refractory mutation system (ARMS™-allele specific amplification). In a further embodiment, the PCR is performed using one or more fluorescently labelled probes or using one or more probes which include a DNA minor groove binder. The presence or absence of a particular SNP allele can also be determined, for example, by sequence analysis.

The nucleic acid sequence method for diagnosis is preferably one which is determined by a method selected from amplification refractory mutation system, restriction fragment length polymorphism and primer extension. In another embodiment, the nucleotide present at each polymorphic position is determined by sequence analysis, such as by dideoxy sequencing.

Preferred mutation detection techniques include ARMS™-allele specific amplification, ALEX™, COPS, Taqman, Molecular Beacons, RFLP, and restriction site based PCR and FRET techniques. Immunoassay techniques are known in the art e.g. A Practical Guide to ELISA by D M Kemeny, Pergamon Press 1991; Principles and Practice of Immunoassay, 2^{nd} edition, C P Price & D J Newman, 1997, published by Stockton Press in USA & Canada and by Macmillan Reference in the United Kingdom.

Particularly preferred methods include ARMS™-allele specific amplification, OLA and RFLP based methods. The allele specific amplification technique known in the art as ARMS™-allele specific amplification is an especially preferred method. ARMS™-allele specific amplification (described in European patent No. EP-B-332435, U.S. Pat. No. 5,595,890 and Newton et al. (Nucleic Acids Research, Vol. 17, p. 2503; 1989)), relies on the complementarity of the 3′ terminal nucleotide of the primer and its template. The 3′ terminal nucleotide of the primer being either complementary or non-complementary to the specific mutation, allele or polymorphism to be detected. There is a selective advantage for primer extension from the primer whose 3′ terminal nucleotide complements the base mutation, allele or polymorphism. Those primers which have a 3′ terminal mismatch with the template sequence severely inhibit or prevent enzymatic primer extension. Polymerase chain reaction or unidirectional primer extension reactions therefore result in product amplification when the 3′ terminal nucleotide of the primer complements that of the template, but not, or at least not efficiently, when the 3′ terminal nucleotide does not complement that of the template.

In a further diagnostic aspect of the invention the presence or absence of variant nucleotides is detected by reference to the loss or gain of, optionally engineered, sites recognised by restriction enzymes. The person of ordinary skill will be able to design and implement diagnostic procedures based on the detection of restriction fragment length polymorphism due to the loss or gain of one or more of the restriction sites due to the presence of a polymorphism.

The invention further provides nucleotide primers, which detect the DRB1 gene polymorphisms of the invention. Such primers can be of any length, for example between 8 and 100 nucleotides in length, but will preferably be between 12 and 50 nucleotides in length, more preferable between 17 and 30 nucleotides in length.

According to another aspect of the present invention there is provided an allele specific primer or probe capable of detecting whether or not the individual has a sequence according to SEQ ID NO: 2 (DQA1*02 allele), or whether the individual has a G nucleotide at rs2858869, a C nucleotide at rs17426385 or a G nucleotide at rs9275141 (positions 101, 101 or 401 of SEQ ID NO: 3, SEQ ID NO: 4 or SEQ ID NO:5, respectively)

An allele specific primer is used, generally together with a constant primer, in an amplification reaction such as a PCR reaction, which provides the discrimination between alleles through selective amplification of one allele at a particular sequence position e.g. as used for ARMS™-allele specific amplification assays. The allele specific primer is preferably 17-50 nucleotides, more preferably about 17-35 nucleotides, more preferably about 17-30 nucleotides.

An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer. Often the nucleotide at the −2 and/or −3 position (relative to the 3′ terminus) is mismatched in order to optimise differential primer binding and preferential extension from the correct allele discriminatory primer only.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1st Edition. If required the primer(s) may be labelled to facilitate detection.

The allele-specific oligonucleotide probe is preferably 17-50 nucleotides, more preferably about 17-35 nucleotides, more preferably about 17-30 nucleotides.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection, such as in Molecular Beacons. Single stranded oligonucleotides corresponding to the polymorphic base in SEQ ID NO: 3, 4 or 5 (positions 101, 101 or 401, respectively) or their complement, could be used as probes to detect the particular polymorphism at the central position. The probe would bind more efficiently to a target sequence that possessed the particular complementary polymorphism base at this central (polymorphism) location than one with a base mismatch.

According to another aspect of the present invention there is provided an allele specific primer or an allele specific oligonucleotide probe capable of detecting the presence of HLA-DRB1*07 and/or HLA-DQA1*02, and/or any of the SNPs: rs2858869, rs17426385, or rs9275141, and its/their use in any of the methods of the present invention.

According to another aspect of the invention there is provided a kit for screening for a genetic predisposition to experience elevated ALAT following ximelagatran administration, which kit comprises:
(i) reagents for detecting the presence of HLA-DRB1*07 or a polymorphism in linkage disequilibrium with D′>0.68 therewith, and optionally,
(ii) means for collecting a biological sample.

In a particular embodiment the polymorphism is in linkage disequilibrium with a D′>0.8. In a further particular embodiment the polymorphism is in linkage disequilibrium with a D′>0.9.

In one embodiment the biological sample is a nucleic acid sample or nucleic acid containing sample. In another embodiment the polymorphism in linkage disequilibrium with D′>0.68 to HLA-DRB1*07 allele is DQA1*02 or the SNPs rs2858869, rs17426385, or rs9275141.

In a particular embodiment the polymorphism is in linkage disequilibrium with a D′>0.8. In a further particular embodiment the polymorphism is in linkage disequilibrium with a D′>0.9.

According to another aspect of the invention there is provided an in vitro diagnostic kit for determining the identity of the alleles DRB1*07, DQA1*02 or the SNPs rs2858869, rs17426385, or rs9275141 in the human DRB1 gene region, said kit comprising components for the determination of the nucleotides present at said gene locations.

In particular embodiments of the invention, the kit components for determining the said SNP include allele-specific amplification primers or allele-specific hybridisation probes capable of determining the identity of the nucleotide base at the SNP location.

According to another aspect of the invention there is provided a kit comprising one or more diagnostic primer(s) and/or one or more allele-specific oligonucleotide probes(s) capable of determining the identity of the nucleotides present at DQA1*02 or any of the SNPs rs2858869, rs17426385, or rs9275141, or a polymorphism in linkage disequilibrium with D′>0.68 therewith, in the human DRB1 gene region.

In a particular embodiment the polymorphism is in linkage disequilibrium with a D′>0.8. In a further particular embodiment the polymorphism is in linkage disequilibrium with a D′>0.9.

The diagnostic kits may comprise appropriate packaging and instructions for use in the methods of the invention. Such kits may further comprise appropriate buffer(s) and polymerase(s) such as thermostable polymerases, for example taq polymerase. Such kits may also comprise companion primers and/or control primers or probes. A companion primer is one that is part of the pair of primers used to perform PCR. Such primer usually complements the template strand precisely.

According to another aspect of the invention there is provided the use of a diagnostic kit capable of determining an individuals' likelihood of experiencing elevated ALAT following ximelagatran administration, by haplotyping the DRB1 gene at least two locations, at least one of which is rs2858869 or exon 2 of the DRB1 gene, for stratifying individuals into particular haplotype groups.

According to another aspect of the invention there is provided the use of a diagnostic kit capable of determining an individual's risk of experiencing elevated ALAT following ximelagatran administration, by haplotyping the DRB1 gene region at least two locations, at least one of which is the DQA1 gene or exon 2 of the DRB1 gene, for stratifying individuals into particular haplotype groups.

One particular application of this is for selecting members from one or more of the stratified groups for inclusion in a clinical trial to determine the efficacy of a drug. In a particular embodiment, the clinical trial is measuring the efficacy of the drug at treating atherosclerosis or an atherosclerosis mediated disease. According to another aspect of the invention there is provided a method for selecting individuals for participation in a clinical trial to assess the effect of ximelagatran administration on ALAT levels in an individual, comprising:
a) individually genotyping the DRB1 gene for the presence of the DRB1*07 allele from a nucleic acid containing sample already isolated from each individual;
b) grouping the individuals according to the particular genotype that each individual belongs to, and
c) selecting individuals from one or more genotype groups for inclusion in the clinical trial.
   The genotype groups will be homozygous positive (individuals with 2 copies of DRB1*07), heterozygous (individuals with one copy of DRB1*07) and homozygous negative (individuals that lack a copy of DRB1*07).

The data generated from the methods and kits of the invention represent a valuable information source with which to characterise individuals in terms of, for example, their likelihood to experience elevated ALAT following ximelagatran administration. These data may be stored in a computer readable medium. The alleles and particular polymorphisms referred to herein are particularly useful as components in databases useful for sequence identity, genome mapping, pharmacogenetics and other search analyses. Generally, the sequence information relating to the nucleic acid sequences and polymorphisms of the invention may be reduced to, converted into or stored in a tangible medium, such as a computer disk, preferably in a computer readable form. For example, chromatographic scan data or peak data, photographic scan or peak data, mass spectrographic data, sequence gel (or other) data.

The computer readable medium may be used, for example, in homology searching, mapping, haplotyping, genotyping or pharmacogenetic analysis. The computer readable medium can be any composition of matter used to store information or data, including, for example, floppy disks, tapes, chips, compact disks, digital disks, video disks, punch cards and hard drives.

The compounds of WO 94/29336 and the prodrug compounds of WO 97/23499 are expected to be useful in those conditions where inhibition of thrombin is required.

In particular, the compounds of WO 97/23499, and ximelagatran in particular, are thus indicated both in the therapeutic and/or prophylactic treatment of thrombosis and hypercoagulability in blood and tissues of animals including man.

It is known that hypercoagulability may lead to thrombo-embolic diseases. Thromboembolic diseases which may be mentioned include: activated protein C resistance, such as the factor V-mutation (factor V Leiden), and inherited or acquired deficiencies in antithrombin III, protein C, protein S, heparin cofactor II. Other conditions known to be associated with hypercoagulability and thrombo-embolic disease include circulating antiphospholipid antibodies (Lupus anticoagulant), homocysteinemi, heparin induced thrombocytopenia and defects in fibrinolysis. The compounds of WO 97/23499, and ximelagatran in particular, are thus indicated both in the therapeutic and/or prophylactic treatment of these conditions.

The compounds of WO 97/23499, and ximelagatran in particular, are further indicated in the treatment of conditions where there is an undesirable excess of thrombin without signs of hypercoagulability, for example in neurodegenerative diseases such as Alzheimer's disease.

Particular disease states which may be mentioned include: the therapeutic and/or prophylactic treatment of venous thrombosis and pulmonary embolism, arterial thrombosis (e.g. in myocardial infarction, unstable angina, thrombosis-based stroke and peripheral arterial thrombosis) and systemic embolism usually from the atrium during arterial fibrillation or from the left ventricle after transmural myocardial infarction.

Moreover, the compounds of WO 97/23499, and ximelagatran in particular, are expected to have utility in prophylaxis of re-occlusion (i.e. thrombosis) after thrombolysis, percutaneous trans-luminal angioplasty (PTA) and coronary bypass operations; the prevention of re-thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis.

In addition to its effects on the coagulation process, thrombin is known to activate a large number of cells (such as neutrophils, fibroblasts, endothelial cells and smooth muscle cells). Therefore, the compounds of WO 97/23499, and ximelagatran in particular, may also be useful for the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosclerosis such as coronary arterial disease, cerebral arterial disease, peripheral arterial disease, reperfusion damage, and restenosis after percutaneous trans-luminal angioplasty (PTA).

Compounds of WO 97/23499, and ximelagatran in particular, that lead to inhibition of trypsin and/or thrombin may also be useful in the treatment of pancreatitis.

According to a further aspect of the present invention, there is provided a method of treatment of a condition where inhibition of thrombin is required which method comprises administration of a therapeutically effective amount of a compound of WO 97/23499, and ximelagatran in particular, or a pharmaceutically acceptable salt thereof, to a person suffering from, or susceptible to such a condition, which person has been previously tested for the presence of an HLA-DRB1*07 allele. In a preferred embodiment, the treatment is applied to individuals that lack an HLA-DRB1*07 allele.

The compounds of WO 97/23499, and ximelagatran in particular, will normally be administered orally, buccally, rectally, dermally, nasally, tracheally, bronchially, by any other parenteral route or via inhalation, in the form of pharmaceutical preparations comprising the prodrug either as a free base, or a pharmaceutical acceptable non-toxic organic or inorganic acid addition salt, in a pharmaceutically acceptable dosage form. Depending upon the disorder and patient to be treated and the route of administration, the compositions may be administered at varying doses.

The compounds of WO 97/23499, and ximelagatran in particular, may also be combined and/or co-administered with any antithrombotic agent with a different mechanism of action, such as the antiplatelet agents acetylsalicylic acid, ticlopidine, clopidogrel, thromboxane receptor and/or synthetase inhibitors, fibrinogen receptor antagonists, prostacyclin mimetics and phosphodiesterase inhibitors and ADP-receptor (P₂T) antagonists.

The compounds of WO 97/23499, and ximelagatran in particular, may further be combined and/or co-administered with thrombolytics such as tissue plasminogen activator (natural or recombinant), streptokinase, urokinase, prourokinase, anisolated streptokinase plasminogen activator complex (ASPAC), animal salivary gland plasminogen activators, and the like, in the treatment of thrombotic diseases, in particular myocardial infarction.

According to a further aspect of WO 97/23499 there are provided suitable pharmaceutical formulations. Suitable daily doses of the compounds of WO 97/23499, and ximelagatran in particular (especially ximelagatran in a form disclosed in WO 00/14110), in therapeutical treatment of humans are about 0.001-100 mg/kg body weight at peroral administration and 0.001-50 mg/kg body weight at parenteral administration.

The compounds of WO 97/23499, and ximelagatran in particular, are inactive per se to thrombin, trypsin and other serine proteases. The compounds thus remain inactive in the gastrointestinal tract and the potential complications experienced by orally administered anticoagulants which are active per se, such as bleeding and indigestion resulting from inhibition of trypsin, may thus be avoided.

Furthermore, local bleeding associated with and after parenteral administration of an active thrombin inhibitor may be avoided by using the compounds of WO 97/23499, and ximelagatran in particular.

Thus, according to another aspect of the present invention there is provided a method of treating a human in need of treatment with ximelagatran, which the method comprises:
i) determining the HLA DRB1*07 status of the human; and,
ii) If the human does not possess DRB1*07, administering an effective amount of the drug ximelagatran.

According to another aspect of the present invention there is provided a method of treating a patient in need of anti-thrombotic treatment comprising:
(a) determining whether or not the patient possesses a copy of HLA-DRB1*07; and,
(b) if the patients does not possess at least one copy of HLA-DRB1*07, treating the patient with an anti-thrombotic agent.
   In a particular embodiment of this aspect of the invention the anti-thrombotic agent is ximelagatran or melagatran.

According to another aspect of the invention there is a provided a method of recommending an anti-thrombotic treatment, the method comprising:
(a) selecting a patient in need of anti-thrombotic treatment whose genome has been identified as lacking HLA-DRB1*07; and recommending that the patient be treated with ximelagatran or melagatran.

According to another aspect of the invention there is a provided a method of treatment comprising:
(a) selecting a patient in need of anti-thrombotic treatment, the patient's genome having been identified as lacking an HLA-DRB1*07; and
(b) treating the patient with ximelagatran.

According to another aspect of the present invention there is provided a pharmaceutical pack comprising ximelagatran and instructions for administration of the drug to humans diagnostically tested for HLA-DRB1*07.

According to another aspect of the invention there is a provided the use of ximelagatran in the manufacture of a medicament for treating patients in need of anti-thrombotic treatment and whose genomes lack one or both copies of HLA-DRB1*07.

According to another aspect of the present invention there is provided use of an antibody capable of selectively binding to DR7 antigen for assessing an individual's likelihood of experiencing elevated ALAT following ximelagatran administration.

Antibodies can be prepared using any suitable method. For example, purified polypeptide may be utilized to prepare specific antibodies. The term “antibodies” is meant to include polyclonal antibodies, monoclonal antibodies, and the various types of antibody constructs such as for example F(ab′)₂, Fab and single chain Fv. Antibodies are defined to be specifically binding if they bind DR7 with a Kₐ of greater than or equal to about 10⁷ M⁻¹. Affinity of binding can be determined using conventional techniques, for example those described by Scatchard et al., Ann. N.Y. Acad. Sci., (1949) 51:660.

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011; 4,902,614; 4,543,439 and 4,411,993; Monoclonal Antibodies, Hybridomas: A New Dimension in Biological Analyses, Plenum Press, Kennett, McKearn, and Bechtol (eds.), (1980).

Monoclonal antibodies for use in the invention can be produced using alternative techniques, such as those described by Alting-Mees et al., “Monoclonal Antibody Expression Libraries: A Rapid Alternative to Hybridomas”, Strategies in Molecular Biology (1990) 3:1-9, which is incorporated herein by reference. Similarly, binding partners can be constructed using recombinant DNA techniques to incorporate the variable regions of a gene that encodes a specific binding antibody. Such a technique is described in Larrick et al., Biotechnology, (1989) 7: 394.

Once isolated and purified, the antibodies may be used to detect the presence of antigen in a sample using established assay protocols, see for example “A Practical Guide to ELISA” by D. M. Kemeny, Pergamon Press, Oxford, England.

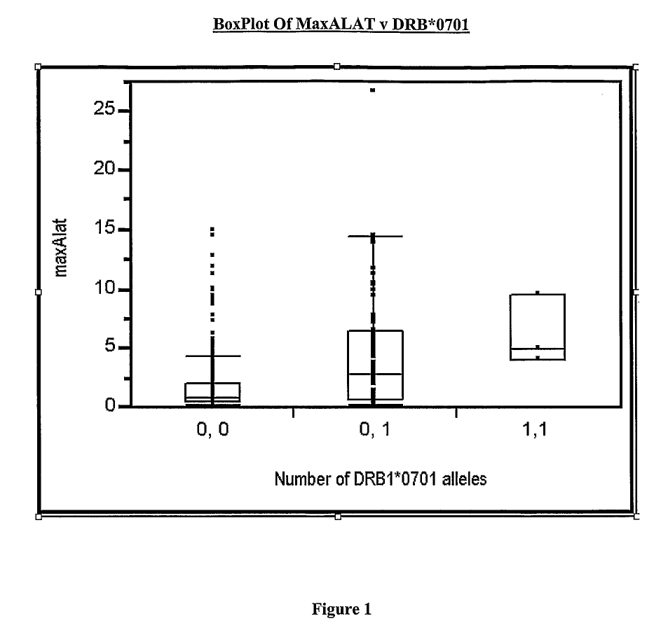

The invention will now be illustrated but not limited by reference to the following non-limiting examples and FIG. 1 which shows a Boxplot of maximum ALAT levels against HLA DRB1*0701 status.

### EXAMPLE 1

In summary, the present invention arose from an open, multicentre, retrospective pharmacogenetic case-control study seeking explanation of ximelagatran's pharmacodynamic effects on transient liver enzymes during long-term treatment. The objective of the study was to retrospectively investigate whether elevated alanine aminotransferase (ALAT) levels during long-term ximelagatran treatment are associated with any specific genetic variations.

### Subject Population

Subjects who had a transient increase of ALAT≧3×ULN and thereafter returned to the baseline level at any time period during days 45-160 of treatment (cases) were compared with subjects (controls) selected from the same studies but without ALAT increase during this period.

In Phases 1 & 2, subjects were selected from males and females who had taken part in one of the long-term treatment ximelagatran studies in the following indications: prevention of stroke in atrial fibrillation; treatment of venous thromboembolism; long-term secondary prevention after treatment of VTE; or, secondary prevention in acute coronary syndromes.

In order to increase the statistical power of the analysis, 2 controls were selected for each identified case. To minimise differences in environmental background and increase the homogeneity of the genetic characteristics, the 2 controls were (as far as operationally possible within each country) matched for weight, age, and sex and, if possible, were chosen from the same investigational site as the corresponding case.

Potential control subjects were identified from the databases of the nominated studies, to be matched for study, country, study site, sex, age, and weight. Where it was not possible to match controls for all the above criteria, matching criteria were prioritised as follows: country and study, sex (where possible), and site (where possible); the difference between case and control age and weight were minimised such that the highest priority controls had least difference between case and control. An algorithm was devised to assign priorities to controls:

Priority=(ABS(100−(100*Control Age/Case Age))+ABS(100−(100*Control weight/Case weight)))/2

Since no clear relationship of ALAT elevation to ximelagatran dose had been identified, it was decided not to match cases and controls for dose, but instead to use dose as a covariate in the statistical model. By not including dose as a criterion for selecting cases and controls, there was greater opportunity to match for other variables.

Subjects who had a transient increase of ALAT≧3×ULN and thereafter returned to the baseline level at any time period during days 45-160 of treatment (cases) were compared with subjects (controls) selected from the same studies but without ALAT increase during this period. In this analysis 74 cases and 169 controls were selected. Case-control status was used as the primary variable for statistical analysis. Max ALAT and AUC in the treatment interval 0-180 days were used for quantitative trait association analysis.

A single blood sample with informed consent was obtained from each of the subjects in the study. DNA was extracted from these samples using standard methodology and thousands of single nucleotide polymorphism (SNP) markers across the genome were genotyped in each sample.

The following standard methods were used for statistical analysis:
Differences in SNP genotype and allele frequencies between cases & controls
ANOVA of differences in max ALAT and AUC between SNP genotype groups
Logistic regression analysis of haplotype frequencies between cases & controls
Standard regression analysis of differences in max ALAT and AUC between haplotypes

The association results for each gene were summarised into a single statistic, p_min, which is simply the minimum p-value across all of the analyses for the gene. SNPs were ranked in terms of lowest p value.

The lowest P value obtained (7.5×10⁻⁶) was for SNP rs2858869, located in non-coding DNA in the 5′ flanking region of DRB1. Hence, genotyping of DRB1, which was known to be highly polymorphic, was performed using standard methodology. In subsequent experiments other markers close to DRB1 were genotyped, including the DQA1 gene and SNPs rs17426385 and rs9275141.

TABLE 1
Details of DRB1*07 and genetic markers that predict the presence of DRB1*07

| | | | | | Allele | | |
|---|---|---|---|---|---|---|---|
| Gene or | | | | | associated | | |
| SNP id | | | | | with | | |
| (HUGO name | | | | | elevated | | D′ with |
| or rs number) | P_min | 5′ flank | SNP | 3′ flank | ALAT | position | DRB1*07 |
| HLA-DRB1 | 9.1 × 10⁻⁶ | N/a | N/a | N/a | DRB1*07 | SEQ01 | — |
| HLA-DQA1 | 1.0 × 10⁻⁵ | N/a | N/a | N/a | DQA1*02 | SEQ02 | 0.98 |
| 2858869 | 7.5 × 10⁻⁶ | AGACT | A/G | GACAT | G | 101 of | 0.85 |
| | | GATAG | | AATGC | | SEQ03 | |
| 17426385 | 7.3 × 10⁻⁸ | CATTT | T/C | TACTTT | C | 101 of | 1.00 |
| | | ACTTA | | ACCT | | SEQ04 | |
| 9275141 | 1.96E−07 | GGTGA | T/G | ACTAC | G | 401 of | 0.69 |
| | | TAGA | | AATCC | | SEQ05 | |

### HLA DRB1 Typing of Samples

Typing of the HLA DRB1 locus was performed using the AlleleSEQR DRB1 Typing kit (Atria Genetics, U.S.A.), according to the manufacturer's instructions. Resultant DNA sequence traces were interpreted using Assign-SBT 3.2.7 software (Conexio Genomics). Approximately 30% of samples gave an ambiguous result, due to a number of heterozygous allelic combinations that result in the same sequence trace. These ambiguities were resolved using the AlleleSEQR HLA-DRB1 GSSP kit (Atria Genetics, U.S.A.), which generates hemizygous sequences from heterozygous PCR products. The kit was used according to the manufacturer's instructions. A DRB1 genotype was assigned to all samples tested.

### Results

DRB1*07 was found in 47% of cases and 17% of controls tested.

TABLE 2
Distribution of DRB1*07 in cases and controls

| | DRB1 genotype* | | |
|---|---|---|---|
| | 0, 0 | 1, 0 | 1, 1 |
| Cases | 39 (53%) | 32 (43%) | 3 (4%) |
| Controls | 108 (83%) | 22 (17%) | 0 (0%) |

*where 1 = DRB1*07 0 = any other DRB1 allele

These results show a highly significant association between DRB1*07 and raised ALAT levels (p=9.1×10⁻⁶, Fisher's exact test). Performing the analysis with high-resolution (specific) alleles did not improve the significance of the analysis, which then showed highly significant association between DRB1*0701 and raised ALAT levels (p=4×10⁻⁵). An analysis of DRB1 alleles versus maximum ALAT in the cases and controls (combined) demonstrated a highly significant association with an overall p-value p<0.0001 (FIG. 1).

### Genotyping of HLA DQA1:

Typing of DQA1 was performed using SSP typing (Dynal AllSet+™ SSP kit). DQA1*02 was found in 48% of cases and 18% of controls tested.

TABLE 3
Distribution of DQA1*02 in cases and controls

| | DQA1 genotype* | | |
|---|---|---|---|
| | 0, 0 | 1, 0 | 1, 1 |
| Cases | 38 (51%) | 33 (45%) | 3 (4%) |
| Controls | 107 (82%) | 23 (18%) | 0 (0%) |

*where 1 = DQA1*02 0 = any other DQA1 allele

These results show a highly significant association between DQA1*02 and raised ALAT levels (p=1.0×10⁻⁵, Fisher's exact test).

### EXAMPLE 2

An additional 10 subjects, treated with ximelagatran, who had a transient increase of ALAT>4×ULN and thereafter returned to the baseline level at any time period during days 45-160 of treatment (cases) were compared with 16 subjects (controls) selected from the same studies but without ALAT increase during this period. None of these subjects had been included in the genetic analysis described in Example 1, and they were all from centres in Sweden (i.e. a genetically homogeneous population). Case-control status was used as the variable for statistical analysis and genetic markers that had been significantly associated in Example 1 were tested for replication (1-sided exact test). The results are shown in Table 4.

TABLE 4
Test for replication of the association between DRB1*07 and markers
in linkage disequilibrium with DRB1*07 and elevated ALAT

| SNP (rs)/allele | P_min (case control) | P_min (case control) |
|---|---|---|
| ID | in EXAMPLE 1 | in Example 2 |
| DRB1*07 | 9.11E−06 | 0.00597 |
| DQA1*02 | 1.30E−05 | 0.00597 |
| 2858869 | 4.29E−04 | 0.0133 |
| 17426385 | 1.02E−07 | 0.000168 |
| 9275141 | 1.96E−07 | 0.353 |

Table 4 shows that, even in such a small sample set, the association between DRB1*07 and elevated ALAT following ximelagatran treatment was significantly replicated. The association between elevated ALAT and three markers in linkage disequilibrium with DRB1*07 (DQA1*02, rs 2858869 and rs 17426385) was also significantly replicated and with the same risk allele as in the original study. The replication of association between elevated ALAT and rs9275141 was not statistically significant, but this may have been due to the small sample set available for study.

In conclusion, these results suggest that determination of an individual's carrier status for the DR7, DRB1*07 or DRB1*0701 alleles can be used to predict the likelihood that an individual will be a case (transient increase of ALAT≧3×ULN). Hence, a test that determined the carrier status of an individual for the DR7, DRB1*07 or DRB1*0701 alleles could be used to optimise the suitability of an individual for ximelagatran treatment with a positive predictive value of 61% and a negative predictive value of 73%, based on available data.

The determination of the presence of the sequence according to SEQ ID NO: 2 (DQA1*02 allele), or whether the individual has a G nucleotide at rs2858869, a C nucleotide at rs17426385 or a G nucleotide at rs9275141 (positions 101, 101 or 401 of SEQ03, SEQ04 or SEQ05, respectively), could also be used to optimise the benefit: risk ratio of an individual for ximelagatran treatment (alleles of DQA1*02, rs2858869, rs17426385, or rs9275141 are in linkage disequilibrium with DRB1*07 with D′ of >0.68).

In a particular embodiment the polymorphism is in linkage disequilibrium with a D′>0.8. In a further particular embodiment the polymorphism is in linkage disequilibrium with a D′>0.9.

1. A method of diagnosis comprising:
a) providing a biological sample from a human identified as being in need of treatment with ximelagatran, wherein the sample contains a nucleic acid encoding DRB1 gene;
b) testing the nucleic acid for the presence, on at least one allele, of either
i) HLA-DRB1*07 (SEQ ID NO: 1, or a sequence with up to 4 encoded amino acid differences provided the encoded allele retains the same antigenic or binding specificity or function as that encoded by SEQ ID NO: 1, or DR7)
ii) an allele of a polymorphism in linkage disequilibrium with a D′>0.68 with (i); and
c) if either (i) or (ii) is found in at least one allele, diagnosing the human as being in the high risk category of having raised ALAT levels after treatment with the ximelagatran.

2. The method as claimed in claim 1, wherein the allele of a polymorphism in linkage disequilibrium with a D′>0.68 with (i) is selected from: SEQ ID NO: 2 (DQA1*02 allele), A>G at position 101 of SEQ ID NO: 3, T>C at position 101 of SEQ ID NO: 4, T>G at position 401 of SEQ ID NO:5.

3. The method as claimed in claims 1 or 2, wherein if in (c) (i) or (ii) is not found in at least one allele the human is diagnosed as being in the low risk category of having raised ALAT levels after treatment with the ximelagatran.

4. A method for sub-typing a human individual according to their risk status of experiencing elevated ALAT following ximelagatran administration comprising the steps of:
a) treating nucleic acid from a sample that has been removed from the individual so as to identify the alleles present at one or more of the DRB1 gene polymorphisms selected from the group consisting of DRB1*07 (or DR7), DQA1*02, rs2858869, rs17426385 and rs9275141
b) assigning the individual to a particular sub-type based on likelihood of experiencing elevated ALAT following ximelagatran administration, according to the nucleotide(s) detected in step a).

5. The method as claimed in claim 4, wherein the presence, on at least one allele, of DRB1*07 (or DR7), or DQA1*02, or a G nucleotide at rs2858869, or a C nucleotide at rs17426385, or a G nucleotide at rs9275141, puts that individual into a high risk sub-type of experiencing elevated ALAT following ximelagatran administration.

6. The method as claimed in claim 4, wherein the absence, on both alleles, of DRB1*07 (or DR7), or DQA1*02, or a G nucleotide at rs2858869, or a C nucleotide at rs17426385, or a G nucleotide at rs9275141, puts that individual into a low risk sub-type of experiencing elevated ALAT following ximelagatran administration

7. (canceled)

8. (canceled)

9. (canceled)

10. (canceled)

11. An in vitro diagnostic kit for screening for a genetic predisposition to elevated ALAT levels following ximelagatran administration, which kit comprises components for determining the identity of the alleles present in or at one or more of: DRB1, DQA1, rs2858869, rs17426385 and rs9275141 in the human DRB1 gene region.

12. The kit as claimed in claim 11, wherein the kit components include allele-specific amplification primers or allele-specific hybridisation probes capable of determining the identity of the nucleotide bases at the polymorphic locations.

13. The kit as claimed in claim 11, wherein the kit components include allele-specific immunological reagents and amplification primers or allele-specific hybridisation probes capable of determining the identity of the alleles at the polymorphic locations.

14. A method of treatment comprising:
(a) selecting a patient in need of anti-thrombotic treatment, the patient's genome having been identified as corresponding to DRB1*07 alleles (according to SEQ ID NO: 1), or an allele in linkage disequilibrium with D′>0.68 therewith, on at least one chromosomal copy; and
(b) treating the patient with a compound that inhibits or blocks thrombin.

15. The method as claimed in claim 14, wherein in step (b) the patient is treated with ximelagatran.

16. A method of treating a human in need of treatment with the drug ximelagatran, which method comprises:
i) determining the absence of DRB1*07 alleles in the human DRB1 gene, or an allele in linkage disequilibrium with D′>0.68 therewith,
ii) determining the status of the human by reference to the alleles present in (i); and,
iii) administering an effective amount of the drug.

17. The method as claimed in claim 16, wherein the allele in linkage disequilibrium with DRB1*07 is selected from: DQA1*02, rs2858869, rs17426385 and rs9275141.

18. (canceled)

19. (canceled)
